(19) United States
(12) Patent Application Publication
(10) Pub. No.: US 2002/0076769 A1
(43) Pub. Date: Jun. 20, 2002

(54) Reduced molecular weight galactomannans oxidized by galactose oxidase

(76) Inventors: Richard Brady, Wilmington, DE (US); H. N. Cheng, Wilmington, DE (US); Alfred Jacques Haandrikman, WX Amersfoort (NL); Allison B. Moore, Newark, DE (US); Pong-Kuen Kuo, Hockessin, DE (US); William T. McNabola, Bear, DE (US); Charles R. Wheeler, Claymont, DE (US); Zu-Feng Xu, Newark, DE (US); Richard James Riehle, Wilmington, DE (US); Tuyen Thanh Nguyen, Wilmington, DE (US); Hielke T. Jeerd de Vries, BF ede (NL); John Arthur Lapre, HB Ede (NL)

Correspondence Address: Gary A. Samuels, Hercules Incorporated, Hercules Plaza, 1313 N. Market Street, Wilmington, DE, 19894-0001, US

(21) Appl. No.: 09/920,694

(22) Filed: Aug. 2, 2001

Related U.S. Application Data
(60) Provisional application No. 60/222,869, filed on Aug. 3, 2000.

(51) Int. Cl. C12P 19/04; C08B 37/00; A61K 31/736

(52) U.S. Cl. 435/101; 514/54; 536/114

(57) ABSTRACT

Presented are compositions of reduced molecular weight galactomannans, particularly guar gum, which have been oxidized by the enzyme galactose oxidase. Further, the invention relates to a process for enzymatically reducing the molecular weight of a galactomannan wherein the galactomannan is simultaneously or subsequently oxidized using galactose oxidase, optionally in combination with other enzymes including peroxidases and or catalases. This process enables production of high concentrations of oxidized galactomannans, which have particular use in the paper making industry.

This application is related to U.S. Provisional patent application Ser. No. 60/222,869, filed Aug. 3, 2000 from which priority is claimed.

### FIELD OF THE INVENTION

This invention relates to reduced molecular weight galactomannans, particularly guar gum, which have been oxidized by galactose oxidase. A particular aspect of the invention additionally relates to a novel process for enzymatically reducing the molecular weight of the galactomannan using mannanase, wherein the galactomannan is simultaneously or subsequently oxidized using galactose oxidase. This preferred aspect of the invention enables the making of novel compositions comprising high concentrations of reduced molecular weight galactomannans which have been enzymatically oxidized by galactose oxidase.

### BACKGROUND OF THE INVENTION

Seed galactomannans, because of their viscous properties, have long found use as thickening agents and binding or colloidal holding agents in a number of fields, including as food additives, commercial lubricants, and paper additives. However, the use of these inherently viscous materials has always been subject to intrinsic limitations because the viscosity of the native galactomannans is too high to permit use of the compounds in any but dilute concentrations. Further, it has traditionally been difficult and or expensive and thus commercially impractical to chemically modify the properties of these compounds because of the need to effectively carry out these reactions at low concentrations. The present invention addresses this need by providing a commercially efficient means to produce high concentrations of chemically modified galactomannans; most particularly, highly concentrated solutions of low molecular weight oxidized guar are provided which exhibit excellent properties of temporary wet strength in paper-making applications.

Oxidation of galactomannans, particularly when achieved enzymatically using galactose oxidase, is known to introduce aldehyde groups on the galactose residues within the galactomannans. It is known further that aldehyde-containing galactomannans, in aqueous solution, tend to form crosslinks. Frollini et al, Carbohydrate Polymers 27 (1995) pp. 129-135, and C. Burke (ed.) Carbohydrate Biotechnology Protocols, (1999), Humana Press, (N.J.) p. 79. Galactomannan compositions are known to be useful in the papermaking industry. For example, see U.S. Pat. Nos. 5,633,300; 5,502,091; 5,338,407 and 5,318,669.

Using galactose oxidase to oxidize the galactomannan gums, especially guar gum has been reported. U.S. Pat. No. 3,297,604 (Germino 1967) discloses galactose-containing polysaccharides which are oxidized chemically or enzymatically with galactose oxidase. U.S. Pat. No. 5,554,745 (Chiu 1996) and U.S. Pat. No. 5,700,917 (Chiu 1997) describe an enzymatic oxidation process using a dual-enzyme system (galactose oxidase and catalase) to convert a cationic guar gum to an aldehyde derivative at the C6 position of the galactose side chain in the guar at 1% solids concentration. The guar gum was not enzymatically degraded prior to the enzymatic oxidation. In fact, efforts were made to preserve the molecular weight of such an oxidized cationic gum. Frollini et al (supra) and C. Burke (supra) reported similar enzymatic oxidation of guar gum. However, none of these disclosures report the oxidation of gum hydrolyzates or a solution of such oxidized gum hydrolyzates at a solids concentration higher than about 1%.

Use of mannanase to hydrolyze galactomannan gums such as guar gum has been practiced for at least five decades. Whistler described in 1950 (Whistler et al, J. of Chemical Society 72 (1950) 4938-4939) enzyme preparations from germinated guar seeds that caused rapid decrease in viscosity of a guar gum solution. McCleary (Carbohydrate Research, 71 (1979) 205-230) used mannanase to hydrolyze guar gum in order to analyze the fine structure of the gum. Japanese patent Hei 10 [1998]-36403 describes cationized decomposed galactomannans useful in the cosmetic industry. Japanese patent Sho 55 [1980]-27797 describes a method for producing low viscosity guar using mannanase. EPA 0 557627 A1 (1992) discloses a method of hydolyzing guar with mannanase to produce a food grade gum, and U.S. Pat. No. 4,693,982 (Carter 1987) discloses a method of treating solid guar gum particles with hydrolytic enzymes to reduce molecular weight and thereby improve solubility.

### SUMMARY OF THE INVENTION

The present invention provides galactomannan compositions having a reduced molecular weight wherein the galactomannans are enzymatically oxidized by galactose oxidase.

The preferred galactomannans include guar, locust bean and tara gum, with guar being most preferred. The preferred reduced molecular weight of the guar will range from about 1,000 to about 500,000, while more preferred ranges are from about 10,000 to 400,000, from about 50,000 to about 350,000 and from about 70,000 to about 350,000, and from about 70,000 to about 150,000 daltons.

The molecular weight of the galactomannans of the invention can be reduced in a number of ways, including with acid treatment, enzymatic treatment and treatment with hydrogen peroxide at high temperature being three preferred methods. One of the most preferred means of reducing the molecular weight of the galactomannan is enzymatically, with mannanase being the most preferred enzyme.

The reduced molecular weight galactomannans of the invention are enzymatically oxidized by galactose oxidase, which acts to oxidize the C6 carbon of the galactose residues of the galactomannan to yield an aldehyde group. The preferred reduced molecular weight, enzymatically oxidized galactomannan is guar, having a preferred range of oxidation of from about 5% up to about 100% of the C6 carbon atoms of the galactose residues being oxidized. More preferred ranges are wherein the galactose oxidase oxidizes from about 15% to about 70% of the galactose C6 carbon atoms, from about 15% to about 60% of the galactose C6 carbon atoms while the most preferred range of oxidation is from about 30% to about 45% of the C6 galactose carbon atoms being oxidized. Optionally, the enzymatic oxidation using galactose oxidase can be carried out in the presence of one or more additional enzyme activities, with catalase activity and peroxidase activity being most preferred.

The reduced molecular weight galactomannans of the invention can be in derivatized form, with cationic derivative groups being most preferred. Derivatization of the galactomannan can take place prior to molecular weight reduction or after.

In a preferred aspect of the invention, the reduced molecular weight, enzymatically oxidized galactomannan is made by a process comprising enzymatic molecular weight reduction using mannanase, wherein the process comprises adding the galactomannan to a prepared solution of mannanase with stirring, and subsequently or simultaneously providing galactose oxidase to oxidize the reduced molecular weight galactomannan. In this method of the invention it is possible to achieve novel compositions comprising enzymatically oxidized galactomannans of reduced molecular weight at high concentrations.

In this process of the invention the preferred galactomannan is guar, which can be made in oxidized form to a preferred concentration range of from about 1.5% to about 80%. More preferred ranges include from about 1.5% to about 20%, with the most preferred range being from about 2% to about 10%. The preferred reduced molecular weight range of the guar in this process of the invention is about 1,000 to about 500,000, with more preferred ranges include about 10,000 to 400,000, from about 50,000 to about 350,000, from about 70,000 to about 350,000, and from about 70,000 to about 150,000 daltons.

The process of the invention yields a preferred range of oxidation of guar including from about 5% up to about 100% of the C6 carbon atoms of the galactose residues being oxidized, with a more preferred range of about 15% to about 70%, with an even more preferred range of about 15% to about 60%, while the most preferred range is from about 30% to about 45%. Additionally, in this process of the invention the galactomannan can be in derivatized form.

### DETAILED DESCRIPTION OF THE INVENTION

The present invention provides new compositions comprising low molecular weight galactomannans, particularly guar, that have been oxidized by galactose oxidase. The invention further provides a preferred method of making the oxidized reduced molecular weight galactomannans using mannanase, wherein the method is capable of producing novel compositions comprising oxidized galactomannans at commercially desirable concentrations exceeding 1.5%.

There are several distinct advantages of the compositions of the inventions. (1) First, these compositions can be made at higher concentrations than can be achieved with galactomannans at their native molecular weight. For guar gum and its derivatives, for example, it is normally difficult to make solutions at much higher than 1% because of high viscosity. Commercially useful gums in solution form can be shipped more conveniently and less expensively at higher concentrations, and are ready to use in solution form. (2) Second, a higher level of enzymatic oxidation at the C6 carbon of galactose can be attained using reduced molecular weight galactomannans because the enzyme oxidation is less inhibited by gel formation. For neutral guar gum at 1%, for example, oxidation beyond 20% is difficult because gel formation limits the oxidation. In contrast, a reduced molecular weight guar at 1% (and higher) can be enzymatically oxidized up to a level of more than 40%. (3) A third advantage is that a liquid gum product with good enzymatic oxidation can be achieved at high concentrations. For example, native guar (molecular weight approximately 2 million) at 1% concentration and 20% oxidation of galactose C6 is a gel, whereas guar at 70,000 molecular weight and 5% concentration is a flowable liquid at up to 35-40% oxidation. (4) And finally, the lower molecular weight of the compositions of the invention provides better wet strength decay in temporary wet strength applications in paper. High initial wet strengths can be obtained with the low molecular weight compositions, but a particular advantage of the low molecular weight composition is that the wet strength is lost more quickly and to a greater extent on contact with water than for the corresponding high molecular weight oxidized gums.

These compositions can be especially useful, therefore, in a variety of temporary wet strength applications in paper, such as in tissue and towel. For bathroom tissue, for example, good wet strength decay prevents pipes from getting clogged. Other paper uses include situations where it is advantageous to achieve improved dry strength, z-direction tensile, Scott bond, Mullen burst, ring crush, STFI, tensile energy absorption (TEA), fracture toughness, and possibly sizing enhancement. This would include uses in paper coating, liquid packaging board, virgin and recycled linerboard, lightweight coated paper, fine paper, and newsprint. Application of these compositions can be at the wet end or after the wet end, such as at the size press or in a spray application. Other possible uses include cosmetics, oilfield recovery, construction, adhesives, tablet coating, paint, textiles, toys, and removable adhesives.

The galactomannans of the invention are well-known polysaccharide materials generally derived from seed gums. The commercially important galactomannans are locust bean gum, guar gum and tara gum. Galactomannans are structurally linear polysaccharides based on a backbone of β(1-4)-linked D-mannose residues. Single α-D-galactose residues are linked to the mannose chain by C1 via a glycosidic bond to C6 of mannose. The degree of galactose substitution on the mannose backbone varies depending on the botanical source of galactomannan. In locust bean gum, the average galactose to mannose ratio is 1:4; in tara gum the ratio is approximately 1:3; and for guar, the most preferred galactomannan of the invention, the ratio of galactose to mannose is approximately 1:2.

Within the context of the present disclosure Applicants intend to include various derivatized forms of galactomannans within the scope of the invention. Derivatives of the galactomannans are very well known in this art, and many are commercially available. Roy L. Whistler and James N. Bemiller, ed., Industrial Gums; Polysaccharides and Their Derivatives (Third Edition), Academic Press, New York, 1993. For example, the most common commercially available guar derivatives include hydroxypropyl, carboxymethyl, carboxymethyl-hydroxypropyl, and 2-hydroxy-3-(trimethylammonium chloride) propyl. Other common derivatives include hydroxyethyl, ethyl, guar gum phosphates, and mixed derivatives including mixed cationic and anionic (amphoteric).

Also within the context of the present disclosure Applicants intend to include within the scope of the invention galactomannans which have been treated with various wetting and solubility agents. Many such agents are known in this art. For example, galactomannan products can be mixed with glyoxal or borax to reversibly crosslink the surface of the particles and retard hydration. Glyoxalated guar requires pH of 7 or above to hydrate, while borated guar requires pH below 8 for hydration. These and other treated and coated forms of the basic galactomannans are considered to be within the scope of Applicants' invention.

For purposes of clarity in describing the present invention, Applicants use the term ‘reduced molecular weight’ of the various galactomannans to refer to a galactomannan which exists in a form having an average molecular weight which is a fraction of its native molecular weight. For example, for the preferred galactomannans of the invention; guar, locust bean and tara, the reduced molecular weight refers to a value which is approximately one half or less than the native molecular weight. Locust bean (carob) gum has a native molecular weight generally reported to be in the range of about 300,000 to 360,000 daltons. The preferred reduced molecular weight range of the compositions of the invention for locust is about 1,000 up to about 150,000 daltons. The most preferred galactomannan of the invention, guar, is known to have a native molecular weight of approximately 2,000,000 daltons. The preferred reduced molecular weight range of the compositions of the invention for guar is from about 1,000 to about 500,000 daltons. More preferred molecular weight ranges for guar include about 10,000 to about 400,000 and from about 50,000 to about 350,000 and from about 70,000 to about 350,000, and from about 70,000 to about 150,000 daltons. Another preferred galactomannan of the invention is tara gum. Definitive molecular weight ranges for native tara gum have not been reported, but it is believed that the native molecular weight is between the value for native guar and native locust bean. Thus, with respect to tara gum in the instant invention, the term reduced molecular weight would refer generally to a range of about 1000 daltons up to a value representing about one half of tara gum's native molecular weight.

Within the context of describing molecular weight of galactomannans for the present disclosure, the term molecular weight refers to the weight average. More particularly, the weight average molecular weight refers to a value which is measured by size exclusion chromatography analysis (SEC) using a calibration derived from narrow distribution polyethylene oxide (PEO) and polyethylene glycol (PEG) molecular weight standards.

Also, it is within the scope of Applicants' invention for an individual galactomannan composition to comprise more than a single botanical species of galactomannan. In some commercial applications for these gums, final characteristics of the compositions can be improved and or tailored to specific purposes by using a mixture of one of more galactomannan. Further, the oxidized reduced molecular weight galactomannan compositions of the invention may comprise additional ingredients, as appropriate and advantageous for the intended purpose of the material. Many additives useful in galactomannan compositions are well known in these arts, including, for example bentonite, alum, starch, cationic polymers, sizing agents, wet strength additives, debonder, defoamers and biocides, any of which might be used to impart additional characteristics for a particular intended purpose of a composition of the invention.

The molecular weight of the galactomannans can be reduced by a variety of methods, including acid treatment, enzyme treatment, and heating with hydrogen peroxide. These methods are well known in this art. For example, acid treatment and heating with hydrogen peroxide are methods known to reduce the molecular weight of the galactomannans; Roy L. Whistler and James N. Bemiller, ed., Industrial Gums; Polysaccharides and Their Derivatives (Third Addition), Academic Press, New York, 1993; and U.S. Pat. No. 5,480,984. Commercial preparations of reduced molecular weight galactomannans that are made by these methods are also available; for example, Galactosol® 30M1F (Hercules, Inc., Wilmington, Del.).

A particularly preferred method of reducing the molecular weight of galactomannan is accomplished using the enzyme mannanase. Mannanase, which has been well characterized in the art, is known to hydrolyze mannans (mannan endo-1, 4-β-mannosidase E.C. 3.2.1.78) wherein the endo-mannanase randomly cleaves 1,4-β-D mannosidic linkage in mannans. See, for example, European Patent Application 0 557 627A1 or McCleary, Carbohydrate Research 71 (1979), pp. 205-230. Mannanase activity can be provided in the form of purified mannanase enzyme, or alternatively, mannanase activity can be provided by using one of the commercial preparations of hemicellulases or cellulases which are known to contain mannanase activity. Examples of such commercially available preparations include Hemicellulase GM™ sold by Amano; Enzebo™ cellulase CRX sold by Enzyme Development Corp. of N.Y.; and Gamanase 1.OL sold by Novo Nordisk.

The compositions of Applicants' invention comprise galactomannan having reduced molecular weight wherein the galactomannan is enzymatically oxidized by galactose oxidase. In a well-characterized reaction mechanism, galactose oxidase is known to specifically oxidize the C6 carbon atom of the galactose residues, wherein the alcohol OH group is oxidized to an aldehyde C=O group. Mazur, A. W. ACS Symposium Series, 466 (1991) 99; U.S. Pat. No. 3,297,604 (Germino); and Knowles, P. F. and Ito,N., Perspectives in BioOrganic Chem., Vol.2,207-244, JAI Press LTD (1993). Galactose oxidase may be produced by the fungus Dactylium dendroides, recently renamed Fusarium sp., and has been given the E. C. Number 1. 1.3.9. Within the context of the compositions of Applicants' invention, oxidation of the C6 of galactose by galactose oxidase is accomplished on about 5% up to about 100% of the C6 carbon atoms on the galactose residues. More preferred ranges of oxidation are about 15% up to about 70%; about 15% up to about 60%; with a range of about 30% up to about 45% being most preferred.

The oxidation process can be accomplished using an effective amount of the single enzyme galactose oxidase, however, in a preferred aspect the oxidation reaction can be improved by incorporating a catalase activity and or a peroxidase activity in the galactose oxidase reaction mixture. The presence of either or both of these additional activities can improve the effectiveness of the oxidation reaction, and enables effective oxidation more efficiently and less expensively when commercial quantities of oxidized galactomannan are desired. The increased catalytic activity of galactose oxidase in the presence of a peroxidase and catalase has been shown by Radin, et al., in The Use of Galactose Oxidase_in Lipid Labeling, J. Lipid Res., Vol. 22:536-541, (1981). Applicant has discovered, with respect to the oxidation of galactomannans, that the activity level of galactose oxidase can be increased, i.e., it can be continuously activated, by carrying out the reaction in the presence of a one-electron oxidant such as peroxidase or laccase, together with a hydrogen peroxide remover such as catalase. Galactose oxidase in combination with catalase has been reported in the oxidation of galactomannans, but Applicants have improved this reaction by the addition of a peroxidase activity, wherein surprisingly, the oxidation reaction becomes more economically efficient for large scale commercial applications, even when the three-enzyme system is used.

This separate invention regarding improving the activity level of galactose oxidase by the addition of a one-electron oxidant to continually activate the galactose oxidase, in the presence additionally of a hydrogen peroxide remover to decompose the hydrogen peroxide which is formed as a coproduct in the oxidation of alcohols, is the subject of a separate commonly-owned and concurrently-filed patent application.

A preferred embodiment of Applicants' invention is a process for making a composition comprising galactomannan at a concentration of at least about 1.5%, wherein the galactomannan is enzymatically hydrolyzed by mannanase and oxidized by galactose oxidase to yield an aldehyde group on at least about 5% up to about 100% of the C6 carbon atoms of the galactose residues. The process comprises preparing a solution of an effective concentration of mannanase, and slowly adding to that solution, while stirring or otherwise agitating the solution, galactomannan to a concentration of at least about 1.5% up to about 80%. Then, with continued stirring or agitation, an effective amount of galactose oxidase and a source of oxygen are added. Optionally, this last step wherein galactose oxidase is added can be carried out in the presence of one or more additional activity components including a catalase activity and or a peroxidase activity.

In another aspect of Applicants' process, the reduction of molecular weight of the galactomannan using mannanase can be carried out simultaneously with the oxidation of the galactomannan. In this aspect, the process comprises preparing a solution comprising effective amounts of mannanase and galactose oxidase, and a source of oxygen, and slowing adding to this solution, with continued stirring or other agitation, galactomannan to a concentration of at least about 1.5% up to about 80%. In this aspect also, optionally, the oxidation using galactose oxidase can be carried out in the presence of one or more additional activities including a catalase activity and or a peroxidase activity.

One important consideration in the process of the invention will be the form in which the galactomannan is added to the mannanase solution. Galactomannans exist in a number of solid, particulate and slurry forms, well known in this art. A preferred technique for the present invention is that galactomannan is added to the mannanase solution in the form of particles. This method allows for putting the galactomannan into solution in a highly concentrated form without rapid viscosity increase that will impair the molecular weight reduction, and without the production of visible “grits” in the final solution product. Galactomannan particle size should be selected carefully. For example, using guar, if the guar particles are too fine, the viscosity of the water-soluble gum will increase so rapidly that the dispersion and solubilization of the gum at high concentration will be virtually impossible and impractical at industrial production scale. If the guar particles are too coarse, the final product will be heterogeneous and have visible large particles (“grits”). The preferred particle size range for a guar gum, for example, is between about 40 to about 250 mesh, more preferably between about 60 to about 200 mesh, and most preferably between about 80 to about 200 mesh (75-180 μm). Given these parameters, the preferred particle sizes of other galactomannans are easily determined empirically, depending upon the desired final characteristics of the intended solution and the properties of the starting galactomannan. Coarsely ground gum or the dehulled seeds from which the gum is obtained, e.g., guar splits, can also be used, but mechanical homogenization may be needed to eliminate visible particles in the finished product.

Another important aspect of Applicants' process is the step of adding the galactomannan to the mannanase solution, instead of adding the mannanase to the galactomannan, as is traditionally done. By adding the galactomannan into the enzyme-containing solution, while stirring or otherwise agitating, the enzyme is able to effectively degrade the galactomannan as the galactomannan is added while continually lowering its viscosity. This aspect of the process allows for hydrolyzing guar, for example, up to a high concentration in solution without problematic lumping or difficulty of mixing due to the otherwise rapid viscosity build-up that is traditionally experienced in the process of solubilizing galactomannans. If one tries to disperse the galactomannan into solution without controlling the particle size and or having the mannanase present in solution prior to addition of the galactomannan, it will be very difficult and impractical to make even a 1.5% galactomannan solution at large scale. Applicants have discovered that if proper consideration is not given to particle size and manner of addition of the galactomannan to the mannanase solution, it will not be possible or practical to carry out the enzymatic oxidation reaction at high concentrations of galactomannan. One skilled in the art could resort to making low molecular weight low viscosity gum hydrolyzates at normal solids concentration (0.5-1.5%), then using spray-drying or alcohol precipitation methods to obtain powered hydrolyzates before re-dissolving it at higher concentration, but such processes are cumbersome and expensive for commercial production.

In Applicants' process any galactomannan can be used. Locust bean, tara and guar gums are preferred; with guar gum being the most preferred. As discussed earlier, the galactomannan can be in native form, or it can be used in derivatized form, and or additionally treated to alter the wettability and solubility aspects of the gum. If the starting galactomannan has been treated or coated to improve its wettability or solubility characteristics, the form of the galactomannan which is added to the mannanase solution will be adjusted accordingly, which adjustments are easily determined empirically within the parameters of the invention. For example, if guar is selected as the galactomannan to be reduced and oxidized and the starting guar particles are in a form coated with borate, the particle size and rate of addition of the guar will be less critical. The rate of addition of the galactomannan to the mannanase and the form of stirring or agitation of the solution while the galactomannan is added are also important aspects of this step of the process. Typically, effective stirring and rate of galactomannan addition are adjusted easily within the parameters of the invention to prevent lumping during addition of the galactomannan to the enzyme solution.

In the process of the invention the molecular weight, the degree of the polymerization and the viscosity of the galactomannan are reduced to desired levels in order to accommodate the subsequent or simultaneous enzymatic oxidation reaction at high galactomannan concentrations, and to meet the performance requirements in the desired application. When using guar in the process of the invention, for example, the reduced molecular weight of the hydrolyzed guar is preferably about 1,000 to 500,000 daltons, more preferably about 10,000 to about 400,000 daltons or from about 50,000 to about 350,000 or from about 70,000 to about 350,000, or from about 70,000 to about 150,000 daltons. If other galactomannans are used in the process of the invention, their reduced molecular weight after treatment with mannanase will be approximately one half or less than the starting native molecular weight of the selected galactomannan.

The process of the invention carries out molecular weight reduction of the starting galactomannan using mannanase mannan endo-1,4-β-mannosidase E.C.3.2.1.78 hydrolysis. This reaction, which has been well studied in the art, is typically carried out at ambient temperature up to 80 degrees C., and in a pH range of about 3 to about 9. The effective concentration range of the mannanase will be adjusted dependent upon the desired final molecular weight and desired final concentration of the selected galactomannan. Cost and time are also factors to be considered. A convenient concentration of mannanase will be approximately 1000 units per gram of galactomannan. The mannanase of the process is essentially free of galactose side chain cleaving α-galactosidase and exo-mannanase activity. After the molecular weight range of the starting galactomannan is reduced to the desired molecular weight range, the mannanase is deactivated by conventional methods such as heat to prevent uncontrolled hydrolytic reaction.

The next step in the process of the invention, (which step can also be carried out simultaneously with the mannanase molecular weight reduction in one aspect of the invention), is the enzymatic oxidation of the galactomannan using galactose oxidase. The term galactose oxidase, for purposes of the present invention, means that enzyme classified as E.C. No. 1. 1.3.9 and those enzymes which function in a substantially similar manner, including, for example, glyoxal oxidase, (CAS Registration No. 109301-01-1). Also included are all enzymes, including those obtained through any form of genetic manipulation, with a catalytic domain which is substantially homologous with galactose oxidase or glyoxal oxidase. As used herein, the term galactose oxidase includes each of the three known oxidative states of galactose oxidase. Galactose oxidase has an approximate molecular weight of 68,000. Knowles and N. Ito, Perspectives in BioOrganic Chemistry, 1993, 2:207-241. As previously stated, enzymatic oxidation is optionally carried out in the presence of a hydrogen peroxide remover such as catalase enzyme, and or a one-electron oxidant such as peroxidase enzyme, which act to assist the galactose oxidase to convert the hydroxyl (—OH) groups at the C6 carbon on the galactose in the galactomannan to hexodialdose (aldehyde).

The concentration of galactose oxidase required in the reaction depends upon the desired characteristics of the finally oxidized galactomannan. In most commercial applications, there will be no upper limit to the amount of enzyme present. However, there will usually be a minimum amount of galactose oxidase that must be present to achieve the desired level of oxidation. Cost and time are factors to be considered. Generally, however, the concentration of galactose oxidase in the aqueous mixture is greater than 1 IU, and more preferably greater than 50 IU per gram of galactomannan.

The term hydrogen peroxide remover, as used herein, is intended to include substances which remove or break-down hydrogen peroxide. It is believed that high levels of hydrogen peroxide may damage the protein structure of galactose oxidase and may inhibit or slow down the galactose oxidase reaction. Accordingly, it is beneficial to maintain the hydrogen peroxide concentration in the reaction medium as low as possible. For purposes of the present invention, hydrogen peroxide removers include, without limitation, catalases.

The term one-electron oxidant, as used herein, is intended to include one-electron oxidants alone and or in combination. A one-electron oxidant, within the scope of the invention, means a substance capable of transforming the inactive form of galactose oxidase to its active form. One-electron oxidant, as used herein, includes for example, the enzymes horseradish peroxidase, soybean peroxidase and laccases. Chemical one-electron oxidants include all chemical one-electron oxidants which are capable of converting the semi or inactive form of galactose oxidase to its active form, including, by way of nonlimiting example, ferricyanide, H₂IrCl₆ [Co(phen)₃]³⁻, [Co(dipic)₂]⁻.

The active oxidized form of galactose oxidase contains a tyrosine radical in the active site. If enzymes are used as one-electron oxidants, only catalytic amounts of the enzyme are required, because the stoichiometic oxidant (either oxygen or hydrogen peroxide) is already present in the reaction mixture in sufficient concentrations. If chemical one-electron oxidants are used, they have to be added in at least stoichiometic amounts, preferably in excess with regards to galactose oxidase.

In addition to maintaining the concentration of hydrogen peroxide at a low level to protect the galactose oxidase (and any other enzymes that may be present, including the one-electron oxidants), the hydrogen peroxide remover can also play a role in providing the molecular oxygen that is needed by galactose oxidase to carry out the oxidation reaction. Galactose oxidase converts the oxidizable galactose type of alcohol configuration to the corresponding aldehyde group (thus producing oxidized galactose). Alternatively, it is known in the art to provide the oxygen via aeration techniques, including bubbling oxygen or air through the solution.

In accordance with the present invention, however, the necessary amount of oxygen may be provided by adding hydrogen peroxide to the catalase containing reaction mixture, wherein the catalase breaks down the hydrogen peroxide into water and oxygen. The addition of oxygen to the reaction mixture by this method is more efficient because it avoids the oxygen transfer from the gas to the liquid phase. Preferably, to prevent the breakdown of the galactose oxidase and or other enzymes present in the system, the hydrogen peroxide is gradually added to the reaction mixture. For optimum oxidation conditions, the addition velocity of the hydrogen peroxide solution is controlled in such a way that the dissolved oxygen concentration in the reaction mixture is maintained, or substantially maintained, at a consistent level. Preferably, the dissolved oxygen concentration is present at saturated or substantially saturated levels.

If a three-enzyme system is used to oxidize the galactomannan substrates (i.e. a galactose oxidase, a catalase and a peroxidase), an appropriate concentration of the enzymes can be determined based on individual reaction parameters. For example, the concentration of the hydrogen peroxide remover (such as catalase E.C. 1.11.1.6) is measured relative to the concentration of galactose oxidase which is used, wherein catalase is present in a ratio greater than 0.1 IU to 1 IU of galactose oxidase. A greater ratio of catalase to galactose oxidase is more preferred, such as up to 10:1 (catalase: galactose oxidase). An appropriate concentration of the one-electron oxidant (such as peroxidase E.C. 1.11.1.7) can also be determined as a ratio between the peroxidase and the galactose oxidase; with a ratio of at least 0.005 IU of soybean peroxidase present per 1 IU of galactose oxidase being preferred. A greater ratio of soybean peroxidase to galactose oxidase is more preferred, such as up to 0.1:1.

Due to a lower viscosity of the hydrolyzed gum which can be achieved in the process of Applicants' invention, it is technically feasible and commercially viable, for example, to enzymatically oxidize up to about 80% available hydroxyl (—OH) group at the C6 carbon of the galactose in guar to aldehyde at up to about a 20% guar concentration. The finished product is a liquid or a “pumpable” gel. In contrast, a galactomannan gum of normal molecular weight can be oxidized only up to approximately 50% aldehyde conversion at only up to about 1% concentration, and the resulting product would be a firm gel that is difficult to disperse in water for most commercial applications. Such a product is not economically feasible and not easy to use. The typical aldehyde conversion for guar in the present process for example, is about 5% up to about 100%, wherein more preferred ranges are about 15% up to about 70%, or about 15% up to about 60% and the most preferred range is about 30% to about 45%. Similar ranges of oxidation are obtainable using other galactomannans of the invention.

In another aspect of the process of the invention the hydrolysis and oxidation reactions can take place simultaneously in one reactor. The amount of mannanase, and optionally catalase and or peroxidase, can be carefully formulated to produce a galactomannan product of desired molecular weight range, degree of aldehyde conversion, and solids concentration. This simultaneous hydrolysis and oxidation reaction can be used to maximize the aldehyde formation at a given molecular weight and galactomannan concentration. When practicing this aspect of the invention, the desired minimum molecular weight and a maximum degree of aldehyde conversion of the derivatized guar can be obtained, within the parameters of the invention which are easily optimized empirically as provided herein by those skilled in the art.

Applicants have discovered that the oxidized gum hydrolyzates of the invention have outstanding functionality in such applications as paper dry or wet strength and exhibit surprisingly rapid decay in water.

In the disclosure of the present invention, all references cited are incorporated herein in their entirety.

The following examples are provided as illustrative in nature only, and are not intended to limit the scope of the teachings provided herein.

### EXAMPLES

For Examples 1 through 21, the paper performance of the oxidized guars was tested in laboratory handsheets. Handsheets were made on a Noble and Wood Sheet Machine (Noble and Wood Machine Co., Hoosick Falls, N.Y.) using standard hard water. Standard hard water (50 ppm alkalinity and 100 ppm hardness) was made by mixing deionized water with CaCl₂ and NaHCO₃. Control of pH was achieved by using NaOH or H²SO₄. Bleached kraft pulp (50% hardwood/50% softwood) was beaten to a Canadian Standard Freeness of about 450 at a consistency of 2.5 weight %. The beaten pulp was added to the proportioner at a controlled level (2000 ml—to adjust basis weight to near 40 lb/3000 ft²) and diluted to 18 liters with standard hard water. Chemical additions and pH adjustments were made to the proportioner as desired, and with continuous mixing. The pH for all data presented here was adjusted to 5.5, and chemical addition was at 0.5% of the dry weight of pulp.

A clean and wetted 100 mesh screen was placed on the open deckle box, which was then closed. Standard hard water and 920 ml of pulp mixture from the proportioner were then added to the deckle box, and dashed. The water was then drained from the box, and the sheet removed. The sheet was wet pressed between felts with press weights adjusted to give a solids content of 33-35%. The sheet and screen were then placed on a drum dryer, which was adjusted to a temperature of 228-232° F. and throughput time of 50-150 sec. Final sheet moisture contents were 3-5%. Five sheets minimum were tested for each experimental set.

Dry tensile testing was done on the handsheets according to TAPPI Method T494 om-88 (“TAPPI Test Methods”, TAPPI Press, Atlanta, Ga., 1996). Wet tensile testing was done by soaking strips for 10 sec or 30 min in deionized water, removing the strip, padding the surface moisture with a paper towel, and testing immediately on an Instron machine. Molecular weights reported are weight average molecular weight by aqueous size exclusion chromatography (SEC). Aldehyde conversions were found by hydrolyzing the products (diluted to 0.2% guar) in acid, and doing high pressure liquid chromatography (HPLC). The change in mannose/galactose ratios was used to calculate % conversion.

### Examples 1-6

These examples show production of reduced molecular weight cationic oxidized guar compositions and their properties in paper compared to high molecular weight guar.

Cationic guar powder (Cosmedia® C-261, Hercules Inc., Del., USA) was treated with 0. 1N, 0.5N, 1.0N or 1.5N HCl at 50° C. for 5 hr at a guar concentration of 2%. The resulting degraded guars were neutralized to pH 7 and precipitated with 80% IPA (isopropyl alcohol) in water, washed with 80% IPA, and air dried. The degraded guars were then milled to less than 0.5 mm. For the enzyme oxidation, typically 11.0 g dry weight of guar was added to 1061 g of deionized water with stirring. Terminox® 50 L (0.099 ml, Novo Nordisk, Denmark) catalase was then added with stirring at 350 rpm and air sparge. Peroxidase 51004, 0.067 ml (Novo Nordisk, Denmark) was then mixed separately with 16.4 ml of 50 mM potassium phosphate buffer (pH 7.0) and 10.53 g galactose oxidase, 990 IU (International Units), (BioTechnical Resources, Wis., USA). This enzyme mix was then added to the guar solution over 1 hr with continued mixing and air sparge. The reaction was continued at pH 7 for 6 hr at room temperature. The following Table shows the resulting weight average molecular weight (SEC) of the guars, % oxidation by, physical form at 1% guar, and paper handsheet properties.

The results demonstrate that at lower molecular weights, a highly oxidized cationic guar at 1% is liquid rather than a gel. Furthermore, the wet strength decay improves at lower molecular weights. At a molecular weight of near 50,000, one can obtain a liquid product with initial wet strength near that of high molecular weight guar, but with wet strength decay improved over the higher molecular weight, high oxidation (>30%) materials.

### Example 7

This example demonstrates production of cationic oxidized guar at 5% solids, and resulting properties. Cosmedia® C-261 was degraded in 1.0N HCl as in Example 5. Oxidation was carried out as in Examples 1-6, except at 5% guar. The recipe was 25 g dry weight of guar, 423 g deionized water, 0.300 ml Terminox® 50L, 0.052 ml Novo 51004, 25 ml phosphate buffer, and 23.94 g galactose oxidase (90 IU/g guar). The product was a flowable liquid at 21% oxidation with excellent wet strength decay.

| | | | | | Dry ten., lb/in | 10 sec. Wet | 30 min. Wet | |
|---|---|---|---|---|---|---|---|---|
| Example | Sample | Mw | Oxid., % | Form | at 0.5% | ten., lb/in | ten., lb/in | WS decay, % |
| 1 | Cosm. C-261 | 1.58 million | 24 | Gel | 20.7 | 3.0 | 2.10 | 30 |
| 2 | Cosm. C261 | 1.58 million | 40 | Gel | 20.8 | 3.2 | 2.5 | 22 |
| 3 | 0.1 N | 669,000 | 51 | Gel | 24.8 | 4.1 | 3.6 | 13 |
| 4 | 0.5 N | 92,500 | 42 | Liquid | 20.9 | 3.2 | 2.6 | 18 |
| 5 | 1.0 N | 47,500 | 41 | Liquid | 20.3 | 2.9 | 2.0 | 30 |
| 6 | 1.5 N | 17,200 | 38 | Liquid | 18.8 | 1.7 | 0.9 | 46 |

*Blank paper (no additive) gave 17.3 lb/in dry tensile and 0.34 lb/in 10 sec wet tensile
WS is wet strength
Ten is Tensile strength

| | | | | | Dry ten., lb/in | 10 sec. Wet | 30 min. Wet | |
|---|---|---|---|---|---|---|---|---|
| Example | Sample | Mw | Oxid., % | Form | at 0.5% | ten., lb/in | ten., lb/in | WS decay, % |
| 7 | 1.0 N | 47,500 | 21 | Liquid | 18.7 | 2.0 | 1.1 | 46 |

*Blank paper gave 15.4 lb/in dry tensile and 0.50 lb/in 10 sec wet tensile.

### Example 8

This example demonstrates the ability to make lower molecular weight neutral oxidized guar at higher oxidative conversion with near the same enzyme level because of less gel limitation.

Supercol® U neutral guar, MW 2.2 million (Hercules Inc., Del., USA) and Galactasol® 30M1F, MW 360,000 (Hercules Inc., Del., USA) produced by peroxide degradation) were oxidized at 1% guar in a procedure similar to that of Examples 3-6. At a galactose oxidase level of 140 IU/g guar, oxidation after 4 hr for Supercol® U was only 18% vs. 42% for 1% 30M1F at 120 IU/g guar. The much higher viscosity of undegraded guar leads to early gelation and difficult oxidation (diffusion limitations of oxygen and/or enzymes). Lower molecular weights are oxidized more readily.

### Examples 9-21

These examples demonstrate the production and properties of low molecular weight neutral oxidized guar compositions. Molecular weight degradation using acid at 2% or mannanase at 10% guar was done, with oxidations performed at 5% guar. Supercol® GF or Supercol G2-S (Hercules Inc., Del., USA) was used for the molecular weight degradations. Several commercial guars, oxidized at 0.8-1%, are included.

For Example 9, Supercol® U neutral guar was degraded with 1.0N acid as in Example 5, and oxidation was carried out at 5% guar as in Example 7. For Examples 10 and 11, a peroxide-degraded guar, Galactasol® 30M1F, was oxidized at 1% as in Example 8. Example 10 used enzymes at one half the level (galactose oxidase at 60 IU/g guar) compared with Example 11. Example 12 demonstrates use of undegraded Supercol U, as described in Example 8.

For Examples 13-21, molecular weight reduction was done with mannanase at 10% guar. A typical procedure was as follows. Mannanase, 491 microliters (Hemicell®, ChemGen Corp. MD, USA,) was added to 1599.3 g of deionized water at 60° C. Supercol® GF or G2-S was then added to the water over 10-15 min with continuous mixing. The solution was mixed at 60° C. for various times, depending on the desired final molecular weight. The mannanase was then deactivated by heating the mixture to 90° C., holding for 30 min, and then cooled. Oxidations were done as follows: 250 g of deionized water was added to 250 g of degraded guar. The pH of the solution was adjusted to 7.2 using 0.5N NaOH. Stirring was set at 300 rpm and with air sparge. Terminox 50 L catalase (Novo Nordisk, Denmark, 300 microliters), Novo 51004 peroxidase (53 microliters), and galactose oxidase (BioTechnical Resources, WI, USA, from Hansenula, 4.0 g) were then added to the mix. The reaction was carried out at room temperature for 4 hr at 25° C. Samples taken during the reaction and at the end were deactivated by adjusting the pH of the mixture to 4.0 using 0.5N H₂SO₄.

The results demonstrate the ability to make liquid products at high solids and high aldehyde conversion. The reduced molecular weight compositions have significant initial wet strength and much improved wet strength decay over higher molecular weight guar.

| | | | | | Dry ten., | 10 sec. Wet | 30 min. Wet | |
|---|---|---|---|---|---|---|---|---|
| Example | Sample | Mw | Oxid., % | Form | lb/in at 0.5% | ten., lb/in | ten., lb/in | WS decay, % |
| 9* | 1.0 N | ˜50,000 | 18 | Liquid | 18.1 | 2.3 | 1.3 | 45 |
| | Sup. U | | | | | | | |
| 10** | 30M1F | 360,000 | 23 | Gel | 20.2 | 3.1 | 2.4 | 23 |
| 11** | 30M1F | 360,000 | 42 | Gel | 22.2 | 4.2 | 3.5 | 16 |
| 12** | Sup. U | 2.2 million | 18 | Gel | 22.7 | 3.5 | 3.3 | 4 |
| 13* | Sup. GF | 70,000 | 22 | Liquid | 17.9 | 2.1 | 1.2 | 44 |
| 14* | Sup. GF | 70,000 | 38 | Liquid | 18.8 | 2.9 | 1.7 | 40 |
| 15* | Sup. GF | 70,000 | 42 | Gel | 18.7 | 3.3 | 1.9 | 42 |
| 16*** | G2S | 54,800 | 34 | Liquid | 19.5 | 2.4 | 1.3 | 46 |
| 17*** | G2S | 54,800 | 38 | Liquid | 19.4 | 2.8 | 1.4 | 49 |
| 18*** | G2S | 54,800 | 44 | Gel | 20.6 | 3.1 | 2.2 | 28 |
| 19*** | G2S | 31,600 | 23 | Liquid | 18.5 | 1.0 | 0.6 | 45 |
| 20*** | G2S | 31,600 | 33 | Liquid | 17.9 | 1.4 | 0.6 | 62 |
| 21*** | G2S | 31,600 | 40 | Liquid | 18.9 | 1.8 | 0.8 | 55 |

*Blank paper (no additive) gave 15.1-15.4 lb/in dry tensile and 0.5 lb/in 10 sec wet tensile.
**Blank paper gave 17.3-17.4 lb/in dry and 0.3-0.5 lb/in wet.
***Blank paper gave 16.6-16.8 lb/in dry and 0.4-0.5 lb/in wet.

### Example 22

The following is an example of making oxidized guar gum hydrolyzates having a molecular weight of approximately 60,000 daltons with 40% aldehyde conversion at 5% solids concentration.

A guar gum of 80 to 200 mesh particle size was selected. Supercol® G2S (Hercules, Inc. DE USA) was used.

The guar was hydrolyzed under the following conditions:

0.0075 part of mannanase was added to 95 parts of water at 60° C. Without delay and while stirring with an overhead mixer, 5 parts of the guar gum was sprinkled into the water within 5-10 minutes. The reaction was allowed to proceed for about 60 minutes to about 55 cps of Brookfield viscosity (at 25° C., 30 rpm with spindle # 31, and a small sample adapter #13 R vessel). The mannanase was deactivated by rapidly heating to 90° C. within 10 minutes using live steam through the jacket of the reactor, and then held at 90° C. for 30 minutes. The reaction mixture was then cooled to 25° C.

The low molecular weight guar was then oxidized under the following conditions:

The pH of the guar gum hydrolyzates solution was adjusted to 7.0 by adding 1.0% of 0.5 M, pH 7.0 phosphate buffer. The temperature was held at 25° C. The solution was sparged with air at 0.4 volume of air per volume of the guar solution per minutes (vvm), while continually stirring at 200 rpm. 120 units of galactose oxidase (BioTechnical Resources, WI, USA), 600 units of catalase Terminox® Ultra 50 L (Novo Nordisk, Denmark), and 15 units of peroxidase NS51004 (Novo Nordisk, Denmark) per gram of guar hydrolyzates was added. The reaction was allowed to proceed for about 4 hours. The oxidation enzymes were deactivated by lowering the pH to 4.0 using 0.5N H₂SO₄, and 0.02% of potassium sorbate was added to the sample to prevent microbial growth.

The final average molecular weight range of the oxidized guar product was determined based upon viscosity/SEC relationship, and the final extent of oxidation was determined using HPLC.

### Example 23

The following is an instructional example of a process wherein one could make a 10% solution of oxidized guar gum having a molecular weight of 25,000 daltons with 25% aldehyde conversion using a simultaneous four-enzyme process.

1) Adjust the pH of 90 parts of water to 7.0 by adding 1.0% of 0.5 M, pH 7.0 phosphate buffer. Keep the temperature at 50° C. while stirring with an overhead mixer. Sparge the solution with air at 0.4 volume of air per volume of the water per minutes (vvm).

2) Add 0.0075 part of mannanase, 120 units of galactose oxidase, 600 units of catalase, and 15 units of peroxidase per gram of dry guar.

3) Without delay, gradually sprinkle 10 parts of the guar gum Supercol® G2S into the water within 10-20 minutes.

4) Start to decrease the temperature at about 15° C. per hour rate.

5) Let the reaction proceed for about 3 hours.

6) Deactivate the enzyme solution by lowering the pH to 4.0 using 0.5N H₂SO₄.

7) Heat the reaction to 90° C. for 30 minutes, then cool to room temperature.

8) Add 0.02% of potassium sorbate to the sample to prevent microbial growth.

We claim:

1. A composition comprising galactomannan having a reduced molecular weight wherein said galactomannan is enzymatically oxidized by galactose oxidase.

2. The composition of claim 1 wherein the galactomannan is selected from the group consisting of one or more of locust bean, tara and guar.

3. The composition of claim 2 wherein the galactomannan is guar.

4. The composition of claim 3 wherein the molecular weight of the guar is from about 1,000 to about 500,000.

5. The composition of claim 4 wherein the molecular weight of the guar is from about 70,000 to about 350,000.

6. The composition of claim 1 wherein the galactomannan having reduced molecular weight is made by a process selected from the group consisting of acid treatment, enzyme treatment, and hydrogen peroxide treatment at high temperature.

7. The composition of claim 6 wherein the galactomannan having a reduced molecular weight is made by the process of enzyme treatment wherein the enzyme comprises mannanase.

8. The composition of claim 1 wherein the enzymatic oxidation of the galactomannan oxidizes from about 5% up to about 100% of the C6 carbon atoms of the galactose residues within said galactomannan.

9. The composition of claim 8 wherein the enzymatic oxidation of the galactomannan oxidizes from about 15% to about 60% of the C6 carbon atoms of the galactose residues within said galactomannan.

10. The composition of claim 8 or 9 wherein the enzymatic oxidation of the galactomannan is optionally carried out in the presence of additional enzyme selected from the group consisting of one or more of peroxidase and catalase.

11. The composition of claim 1 wherein the galactomannan further comprises one or more derivative groups selected from the group consisting of hydroxypropyl, carboxymethyl, carboxymethyl-hydroxypropyl, 2-hydroxy-3-(trimethylammonium chloride) propyl, hydroxyethyl, ethyl, and phosphate groups.

12. The composition of claim 1 wherein the galactomannan comprises guar; the molecular weight of the galactomannan is from about 1,000 to about 500,000; the galactomannan is made by a process selected from enzyme treatment wherein the enzyme comprises mannanase or by acid treatment; and the enzymatic oxidation of the galactomannan oxidizes from about 5% up to about 100% of the C6 carbon atoms of the galactose residues within said galactomannan.

13. The composition of claim 12 wherein the molecular weight of the guar is from about 70,000 to about 350,000; the guar is made by a process selected from enzyme treatment wherein the enzyme comprises mannanase or by acid treatment; and the enzymatic oxidation of the galactomannan oxidizes from about 15% up to about 70% of the C6 carbon atoms of the galactose residues within said galactomannan.

14. The composition of claim 12 or 13 wherein the galactomannan further comprises one or more cationic derivative groups.

15. The composition of claim 12 or 13 wherein the galactomannan is made by the process of enzyme treatment wherein the enzyme comprises mannanase.

16. The composition of claim 15 further wherein the concentration of the galactomannan is from about 1% to about 80%.

17. A process for making a composition comprising galactomannan at a concentration of at least about 1.5% to about 80%; wherein said galactomannan is enzymatically oxidized by galactose oxidase to yield an aldehyde group on at least about 5% of the C6 carbon atoms of the galactose residues within said galactomannan; said process comprising the steps of
a) preparing a solution comprising an effective amount of mannanase,
b) slowly adding to a), with continued stirring, galactomannan to a concentration of at least about 1.5% to about 80% galactomannan; and
c) adding to b), with continued stirring, an effective amount of galactose oxidase, and a source of oxygen.

18. A process for making a composition comprising galactomannan at a concentration of at least about 1.5% to about 80%; wherein said galactomannan is enzymatically oxidized by galactose oxidase to yield an aldehyde group on at least about 5% of the C6 carbon atoms of the galactose residues within said galactomannan; said process comprising the steps of
a) preparing a solution comprising effective amounts of mannanase and galactose oxidase, and a source of oxygen; and
b) slowly adding to a), with continued stirring, galactomannan to a concentration of at least about 1.5% to about 80% galactomannan.

19. The process of claim 17 further wherein at step c, additional enzyme activities selected from the group consisting of one or more of catalase activity and peroxidase activity are added to the solution.

20. The process of claim 18 further wherein at step a, additional enzyme activities selected from the group consisting of one or more of catalase activity and peroxidase activity are added to the prepared solution.

21. The process of claim 17 or 18 wherein the galactomannan added at step b) is selected from the group consisting of one or more of guar, locust bean and tara.

22. The process of claim 21 wherein the galactomannan added at step b) further comprises one or more of the derivative groups selected from the group consisting of a cationic, anionic, amphoteric, hydroxypropyl, and ethyl group.

23. The process of claim 22 wherein the galactomannan added at step b) further comprises one or more cationic derivative groups.

24. The process of claim 21 wherein the galactomannan is guar.

25. The process of claim 24 wherein at step b) the guar is added to a concentration of at least about 1.5% to about 80%.

26. The process of claim 25 wherein at step b) the guar is added to a concentration of at least about 1.5% to about 20%.

27. The process of claim 24 wherein after step b) the guar has a molecular weight of about 1,000 to 500,000.

28. The process of claim 27 wherein after step b) the guar has a molecular weight of about 70,000 to about 350,000.

29. The process of claim 21 wherein the guar is enzymatically oxidized by galactose oxidase to yield an aldehyde group on about 5% up to about 100% of the C6 carbon atoms of the galactose residues.

30. The process of claim 29 wherein the guar is enzymatically oxidized by galactose oxidase to yield an aldehyde group on about 15% to about 60% of the C6 carbon atoms of the galactose residues.

31. The product of the process of claim 17 or 18.
